(19) United States
(12) Patent Application Publication
(10) Pub. No.: US 2010/0185085 A1
(43) Pub. Date: Jul. 22, 2010

(54) DYNAMIC ULTRASOUND PROCESSING USING OBJECT MOTION CALCULATION

(76) Inventor: James Hamilton, Brighton, MI (US)

Correspondence Address: SCHOX PLC, 500 3rd Street, Suite 515, San Francisco, CA, 94107, US

(21) Appl. No.: 12/625,885

(22) Filed: Nov. 25, 2009

Related U.S. Application Data
(60) Provisional application No. 61/145,710, filed on Jan. 19, 2009.

(51) Int. Cl. A61B 8/00; G06K 9/00

(52) U.S. Cl. 600/437; 382/128

(57) ABSTRACT

A system and method for transforming ultrasound data includes acquiring ultrasound data, calculating object motion from the data, modifying a processing parameter, processing the ultrasound data according to the processing parameter, and outputting the processed ultrasound data. The system and method may additionally include the calculation of a data quality metric that can additionally or alternatively be used with object motion to modify a processing parameter.

### CROSS-REFERENCE TO RELATED APPLICATIONS

This application claims the benefit of U.S. Provisional Application No. 61/145,710, filed 19 Jan. 2009, which is incorporated in its entirety by this reference.

### TECHNICAL FIELD

This invention relates generally to the medical ultrasound processing field, and more specifically to a new and useful system and method of dynamic processing in the medical ultrasound field.

### BRIEF DESCRIPTION OF THE FIGURES

FIG. 1 is a flowchart of a preferred method of dynamic ultrasound processing;

FIG. 2 is a flowchart of various sub-steps of the processing step of the preferred method;

FIGS. 3A, 3B, and 3C are flowcharts of various preferred embodiments with dynamic processing using data quality metrics;

FIGS. 4A and 4B are flowcharts of an alternative method using iterative processing;

FIGS. 5A and 5B are flowcharts of a preferred method of controlling an outside object;

FIGS. 6A and 6B is a flowchart of a preferred embodiment processing ultrasound motion data;

FIG. 7 is a schematic representation of a preferred system of dynamic ultrasound processing; and

FIGS. 8A and 8B are exemplary images of data quality metric based filtering that show an average velocity plot of a region of interest prior to filtering, and that show an average velocity plot after filtering out pixels with data quality indexes less than 0.9, respectively.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Dynamic Processing of Ultrasound Data

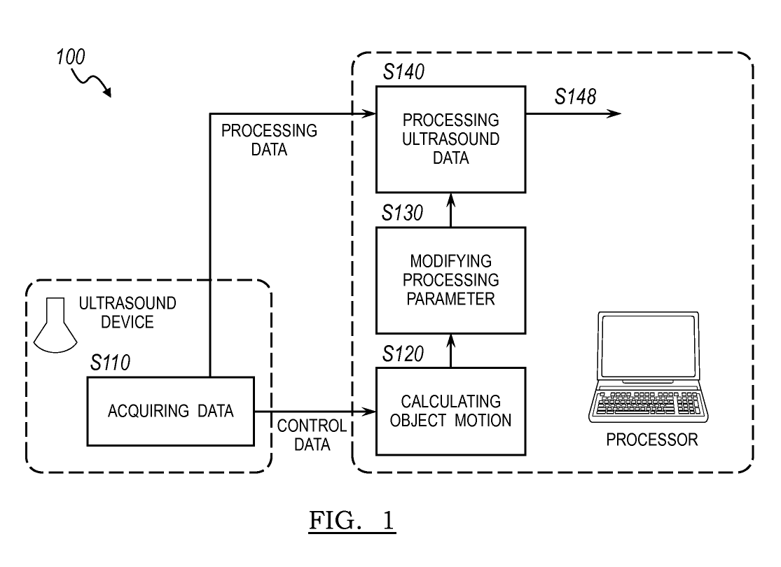

As shown in FIG. 1, the method 100 of dynamic ultrasound processing of the preferred embodiment includes acquiring ultrasound data S110, calculating object motion S120, modifying a processing parameter S130, and processing ultrasound data S140. The method 100 functions to use motion information extracted from an original form of data (e.g., raw ultrasound data) in the transformation (the processing) into a second form of data. The method 100 preferably uses object motion calculations to modify data processing. Additionally, the method 100 may include the use of a data quality metric (DQM) during the dynamic processing. The acquired data may be direct or buffered, and the form of data may be aperture, beamformed, or any suitable form. Alternatively, the object motion calculation and the data processing may each use different sources or forms of ultrasound data.

Step S110 includes acquiring data and, more specifically, acquiring ultrasound data. Step S110 preferably includes the sub-steps of collecting data and preparing data. The step of collecting data functions to collect raw ultrasound data such as from an ultrasound transducer or device storing raw ultrasound data. The raw ultrasound data may be represented by real or complex, demodulated or frequency shifted (e.g., baseband data), or any suitable representation of raw ultrasound data. Preparing data functions to perform preliminary processing to convert the raw data into a suitable form, such as brightness mode (B-mode), motion mode (M-mode), Doppler, or any other suitable form of ultrasound data. The acquired data may alternatively be left as raw ultrasound data, or the acquired data may alternatively be collected in a prepared data format from an outside device. In addition, pre- or post-beamformed data may be acquired. The acquired data may describe any suitable area (either 1D, 2D, 3D), or any suitable geometric description of the inspected material. The acquired data is preferably from an ultrasound device, but may alternatively be any suitable data acquisition system sensitive to motion. The acquired data may alternatively be provided by an intermediary device such as a data storage unit (e.g. hard drive), data buffer, or any suitable device. The acquired data is preferably output as processing data and control data. The processing data is preferably the data that will be processed in Step S140. The control data is preferably used in motion calculation and for processing parameter control. The processing data and control data are preferably in the same format, but may alternatively be in varying forms described above.

Step S120, which includes calculating object motion, functions to analyze the acquired data to detect tissue movement, probe movement, and/or any other motion that affects the acquired data. Object motion preferably includes any motion that affects the acquired data such as tissue motion, tissue deformation, probe movement, and/or any suitable motion. The measured motion may be a measurement of tissue velocity, displacement, acceleration, strain, strain rate, or any suitable characteristic of probe, tissue motion, or tissue deformation. Object motion is preferably calculated using the raw ultrasound data, but may alternatively use any suitable form of ultrasound data. At least two data sets (e.g., data images) acquired at different times are preferably used to calculate 1D, 2D or 3D motion. Speckle tracking is preferably used, but alternatively, Doppler processing, block matching, cross-correlation processing, lateral beam modulation, and/or any suitable method may be used. The motion measurements may additionally be improved and refined using models of tissue motion. The object motion (or motion data) is preferably used as parameter inputs in the modification of processing parameters in Step S130, but may alternatively or additionally be used directly in the processing Step S140.

As mentioned above, speckle tracking is a motion tracking method implemented by tracking the position of a kernel (section) of ultrasound speckles that are a result of ultrasound interference and reflections from scanned objects. The pattern of ultrasound speckles is fairly similar over small motions, which allows for tracking the motion of the speckle kernel within a search window (or region) over time. The search window is preferably a window within which the kernel is expected to be found, assuming normal tissue motion. Preferably, the search window is additionally dependent on the frame rate of the ultrasound data. A smaller search window can be used with a faster frame rate, assuming the same tissue velocity. The size of the kernel affects the resolution of the motion measurements. For example, a smaller kernel will result in higher resolution. Motion from speckle tracking can be calculated with various algorithms such as sum of absolute difference (SAD) or normalized cross correlation.

Step S130, which includes modifying processing parameter(s), functions to utilize object motion calculations to enhance or improve the data processing. The coefficients or control parameters of filters or signal processing operations are preferably adjusted according to parameter inputs that are related to the object motion calculated in Step S120. More preferably, the calculated object motion is used as the parameter inputs to modify the processing parameters. The parameter inputs may additionally or alternatively include other information such as data quality metrics discussed in further detail below. Step S130 may include variations depending on the data processing application. For example, data processing may include tissue motion calculation using speckle tracking. In this case, windows are preferably increased in size and search regions are decreased for the case of speckle tracking in a region of static tissue. Inversely, data windows are preferably decreased in size and search regions are increased for speckle tracking in regions of moving or deforming tissue. Another example of motion controlled data processing is image frame registration. In this case, motion estimates can be used to resample and align B-mode or raw data samples for improved filtering, averaging, or any suitable signal processing. Image resampling coefficients are preferably adjusted to provide frame registration. As another example, the parameter inputs may determine the coefficients, or alternatively, a new coordinate system, used for processing ultrasound data such as when resampling an ultrasound image. The modified processing parameters may additionally be used in the following applications: spatial and temporal sampling of various algorithms, including color-flow (2D Doppler), B-mode, M-mode and image scan conversion; wall filtering for color-flow and Doppler processing; temporal and spatial filters programming (e.g., filter response cut-offs); speckle tracking window size, search size, temporal and spatial sampling; setting parameters of speckle reduction algorithms; and/or any suitable application.

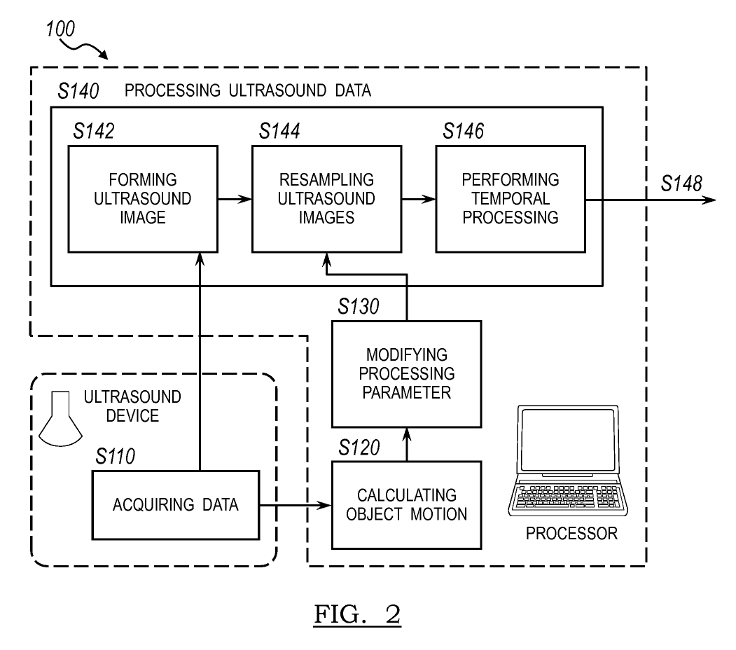

Step S140, which includes processing ultrasound data, functions to transform the acquired data for ultrasound imaging, analysis, or any other suitable goal. The step of processing preferably aids in the detection, measurement, and/or visualizing of image features. After the processing of the ultrasound data is complete, the method preferably proceeds in outputting the processed data (i.e., transformed data) S148. The outputted data may be used for any suitable operation such as being stored, displayed, passed to another device, or any suitable use. The step of processing may be any suitable processing task such as spatial or temporal filtering (e.g., wall filtering for Doppler and color flow imaging), summing, weighting, ordering, sorting, resampling, or other processes and may be designed for any suitable application. Preferably, Step S140 uses the data that was acquired in Step S110 and the parameters that were modified in Step S130. As an example, object motion data (calculated in Step S120) may be used to automatically identify or differentiate between object features such as between blood and tissue in Step S130. Depending on the situation, velocity, strain, or strain-rate calculations or any suitable calculation may be optimized to target only the object features of interest. For example, strain calculations may ignore ultrasound data associated with blood as a way to improve accuracy of tissue deformation measurements. The ultrasound data may be raw ultrasound data (e.g., RF data) or other suitable forms of data such as raw data converted into a suitable form (i.e., pre-processed). Step S140 is preferably performed in real-time on the ultrasound data while the data is being acquired, but may alternatively be performed offline or remotely on saved or buffered data. As shown in FIG. 2, Step S140 preferably includes the sub-steps of forming an ultrasound image S142, resampling of an ultrasound image S144, and performing temporal processing S146. The processing steps of S140 can preferably be performed in any suitable order, and the sub-steps S142, S144, and S146 may all or partially be performed in any suitable combination.

Step S142, which includes forming an ultrasound image, functions to output an ultrasound image from the ultrasound data acquired in Step S110. Ultrasound data from step S110 is preferably converted into a format for processing operations. This step is optional, and is not necessary, such as in the case when the processing step is based upon raw ultrasound data. An ultrasound image is preferably any spatial representation of ultrasound data or data derived from ultrasound signals including raw ultrasound data (i.e., radio-frequency (RF) data images), B-mode images (magnitude or envelope detected images from raw ultrasound data), color Doppler images, power Doppler images, tissue motion images (e.g., velocity and displacement), tissue deformation images (e.g., strain and strain rate) or any suitable images.

Step S144, which includes resampling of an ultrasound image, functions to apply the processing parameters based on the motion data to the processing of the ultrasound data. The resampling is preferably spatially focused, with temporal processing occurring in Step S146, but Step S144 and Step S146 may alternatively be implemented in substantially the same step. Ultrasound image refinements may be made using the motion data as a filter for image processing operations. For example, motion data may be used to identify areas of high tissue velocity and apply image correction (sharpening or focusing) to account for distortion in the image resulting from the motion. Additionally or alternatively, resampling of an ultrasound image may include spatially mapping data, using measurements of the spatial transformation between frames to map data to a common grid. Spatially mapping data preferably includes shifting and additionally warping images by adaptively transforming image frames to a common spatial reference frame. This is preferably used cooperatively with temporal processing of Step S146 to achieve motion compensated frame averaging.

Step S146, which includes performing temporal processing, functions to apply time based processing of successive ultrasound data images. Temporal processing preferably describes the frame-to-frame (i.e., time series) processing. Additionally, the step of performing temporal processing may be performed according to a parameter controlled by the object motion calculation. Temporal processing may include temporal integration, weighted summation (finite impulse response (FIR) filtering), and weighted summation of frame group members with previous temporal processing outputs (infinite impulse response (IIR) filtering). The simple method of frame averaging is described by a FIR filter with constant weighting for each frame. Frame averaging or persistence may be used to reduce noise. Frame averaging is typically performed assuming no motion. Temporal processing can additionally take advantage of spatial mapping of data performed in Step S144 to enhance frame averaging. For example, with a system that acquires data at 20 frames per second (i.e., 50 ms intra-frame time) and an object with an object stability time (i.e., time the underlying object can be considered constant) of 100 ms, only two frames may be averaged or processed without image quality degradation. Using measurements of the spatial transformation between frames, the data can be mapped to a common grid prior to temporal processing to compensate for object motion, providing larger temporal processing windows and ultimately improved image quality from signal to noise increase. In this example, assume the object stability time increases by a factor of 10 (to 1 second) when the probe and object motion is removed. Now, 20 frames can be averaged without degradation, improving the signal to noise ratio by a factor greater than 3 (assuming white noise).

### 2. Dynamic Processing With Data Quality Metric

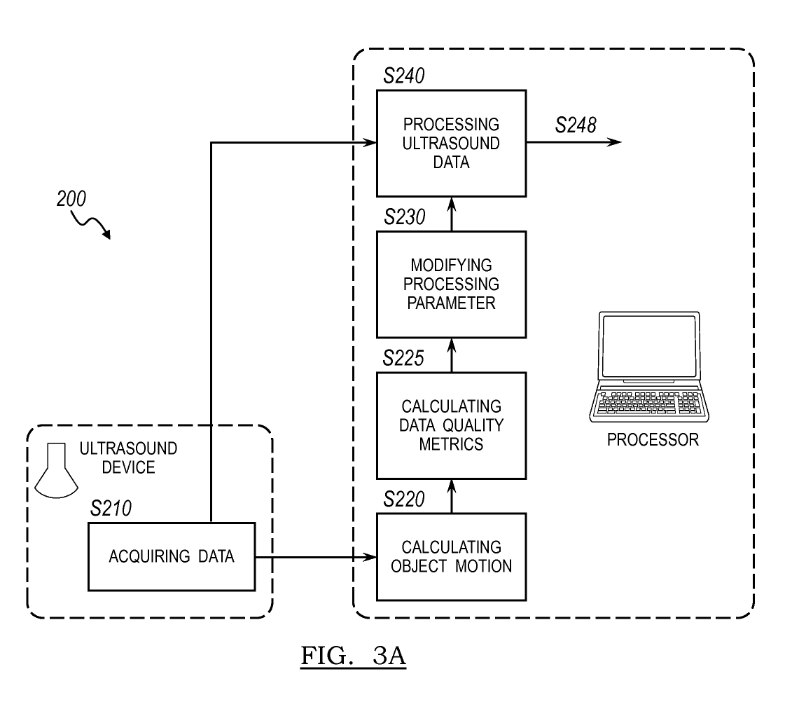

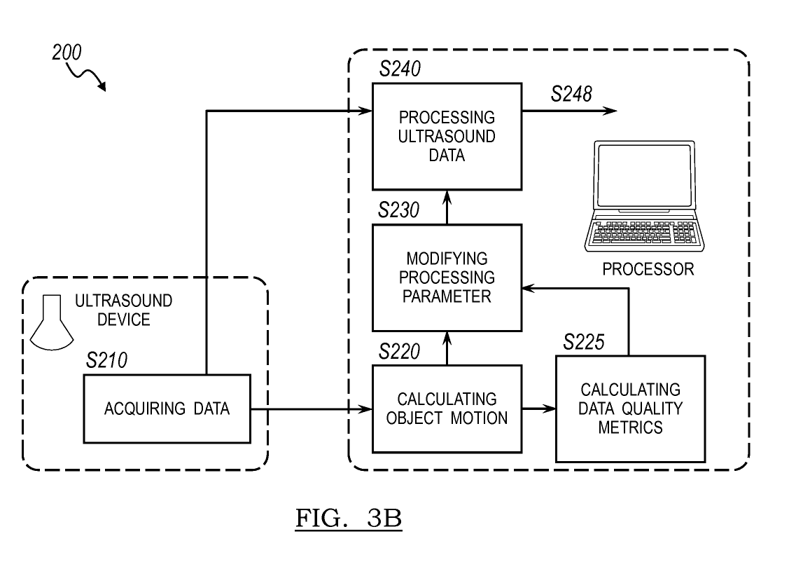

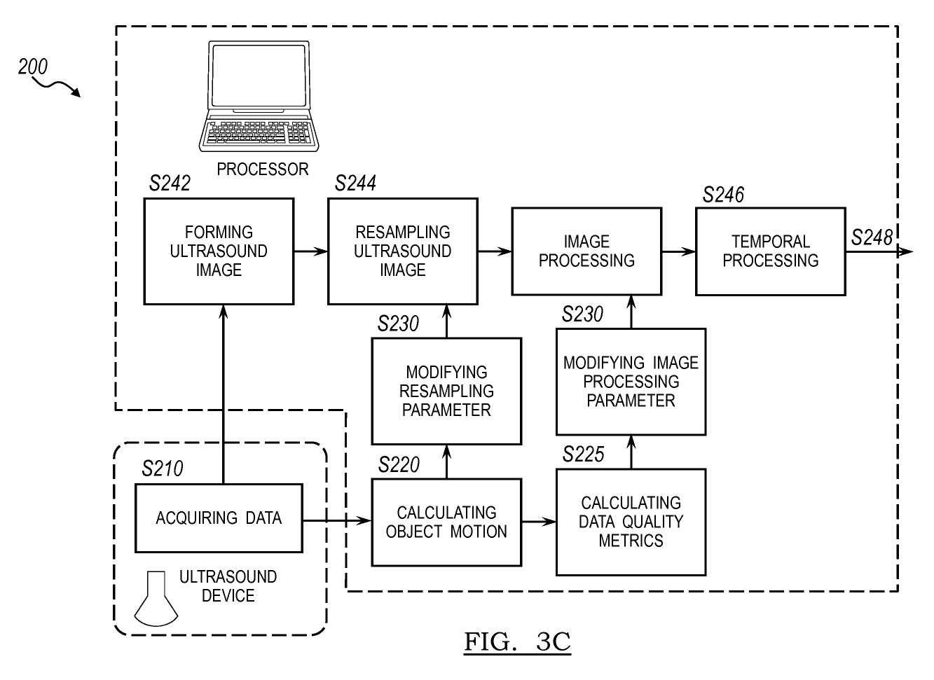

As shown in FIGS. 3A-3C, a method 200 of a second preferred embodiment includes acquiring data S210, calculating object motion S220, calculating data quality metric S225, modifying a processing parameter S230, and processing ultrasound data S240. The method 200 functions to use data quality metric as a discriminatory metric for segmenting and identifying data for processing. The object motion calculations are preferably used as a way of quantifying the quality of data, which can be used to adjust the processing parameters of the ultrasound data. Except as noted below, the steps of acquiring data S210, calculating object motion S220, modifying a processing parameter S230, and processing ultrasound data S240 are substantially similar to Steps S110, S120, S130, and S140 respectively. The additional steps using the DQM may additionally be used with any variations or additional steps of the method of dynamic processing such as those described for the above method 100.

Step S220, which includes calculating object motion, functions to analyze the acquired data to detect tissue movement, probe movement, and/or any other motion that affects the acquired data. Step S220 is preferably substantially similar to Step S120 described above, but Step S220 may additionally contribute to calculating data quality metrics in Step S125. As explained below, speckle tracking performed with normalized cross correlation produces a quantity referred to as data quality index (DQI) that can be used as a DQM. Normalized cross correlation is preferably performed by acquiring ultrasound radio frequency (RF) images or signals before and after deformation of an object. Image regions, or windows, of the images are then tracked between the two acquisitions using the cross-correlation function. The cross-correlation function measures the similarity between two regions as a function of a displacement between the regions. The peak magnitude of the correlation function corresponds to the displacement that maximizes signal matching. This peak value is preferably referred to as the DQI.

Step S225, which includes calculating a data quality metric, functions to aid in the optimization of data processing by determining a value reflecting the quality of the data. The DQM preferably relates to the level of assurance that the data is valid. Data quality metrics are preferably calculated for each sample, sub-set of samples of an image region, and/or for each pixel forming a DQM map. The DQM is preferably obtained from calculations related to tissue velocity, displacement, strain, and/or strain rate, or more specifically, peak correlation, temporal and spatial variation (e.g., derivatives and variance) of tissue displacement, and spatial and temporal variation of correlation magnitude. The data quality metric (DQM) is preferably calculated from a parameter(s) of the speckle tracking method and is more preferably the DQI described above. The DQI is preferably represented on a 0.0 to 1.0 scale where 0.0 represents low quality data and 1.0 represents high quality data. However, any suitable scale may be used. The DQI of data associated with tissue tend to have higher values, than data in areas that contain blood or noise. As is described below, this information can be used in the processing of ultrasound data for segmentation and signal identification. The DQM is preferably used in Step S230 as a parameter input to modify processing parameters.

The DQM may be used individually to modify the processing parameters (FIG. 3A), the DQM may be used cooperatively with calculated object motion to modify processing parameters (FIG. 3B), and/or the DQM and the motion information may be used modify a first and second processing parameter (FIG. 3C).

Step S230, which includes modifying processing parameter(s), functions to utilize object motion calculations and/or DQM to enhance or improve the data processing. The coefficients or control parameters of filters or signal processing operations are preferably adjusted according to the parameter inputs related to object motion measured in Step S220 and/or the DQM of Step S225. The modification of processing parameters may be based directly on DQM (FIG. 3A) and/or calculated object motion (FIG. 1). The modification of the processing parameters may alternatively be based on a combination or of the processing parameters either cooperatively as in FIG. 3B or simultaneously (e.g., individually but in parallel) as in FIG. 3C.

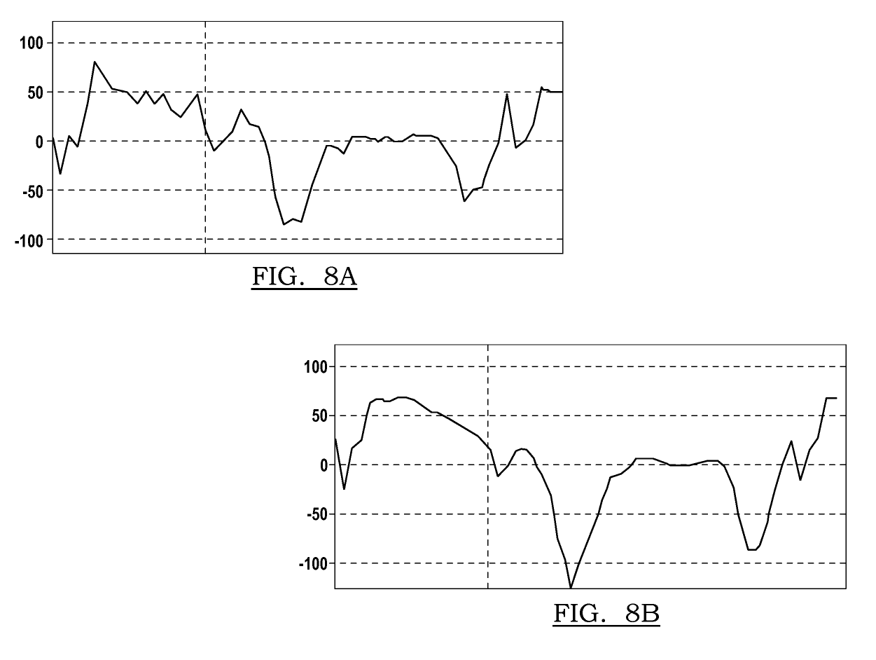

The use of DQM preferably enables a variety of ways to control the processing of data. For example, measurements such as B-mode, velocity, strain, and strain rate may be weighted or sorted (filtered) based on the DQM. The DQM can preferably be used for multiple interpretations. The DQM may be interpreted as a quantized assessment of the quality of the data. Data that is not of high enough quality can be filtered from the ultrasound data. As an example, ultrasound derived velocity measurements for a section of tissue may suffer from noise (shown in FIG. 8a). After filtering velocity measurements to only include measurements with a DQI above 0.9, the noise level is reduced and the measurement improves (shown in FIG. 8b). The DQM may alternatively be interpreted as a tissue identifier. As mentioned above, the DQI can be used to differentiate between types of objects specifically, blood and tissue. Thus, the DQI can be used for segmentation and signal or region identification when processing the ultrasound data. As an example of one application, the DQM, or more specifically the DQI, may be used to determine the blood-to-heart wall boundaries and may be used to identify anatomical structures or features automatically. Processing operations may additionally be optimized by selectively performing processing tasks based on identified features (e.g., tissue or blood). For example, when calculating strain rate of tissue, areas with blood (as indicated by low DQI) can be ignored during the calculation process. The processing operations such as speckle tracking, measuring velocity, measuring strain, measuring strain-rate, changing coordinate systems, or any additional operations are computationally expensive. Additionally, higher frame rates and higher resolution imaging require more processing capabilities. Using DQM to segment ultrasound data or images according to tissue type, tissue specific processing operations can be used to reduce processing requirements for computationally expensive processes. In this variation, computational expensive processes are performed for data of interest. Data of less interest may receive a different process or a lower resolution process to reduce the computational cost.

Step S240, which includes processing ultrasound data, functions to transform the acquired data for ultrasound imaging, analysis, or any suitable goal. The processing of ultrasound data preferably uses the modified processing parameters provided in Step S230. Preferably, Step S240 uses the data that was acquired in Step S210 and the parameters that were modified in Step S230. After the processing of the ultrasound data is complete, method preferably proceeds in outputting the processed data (i.e., transformed data) S248. The outputted data may be used for any suitable operation such as being stored, displayed, passed to another device, or any suitable use. The processing of ultrasound data may include multiple sub-steps as described for Step S140, and modified processing parameters based on motion information and/or DQM may be used for any of these sub-steps. As shown in FIG. 3C a first sub-step of processing the ultrasound data (e.g., resampling an ultrasound image) may be controlled by a first processing parameter, where the first processing parameter is determined by the calculated object motion. A second sub-step of processing the ultrasound data (e.g., image processing) may be controlled by a second processing parameter, where the second processing parameter is determined by the DQM.

### 3. Dynamic Processing With Iteration

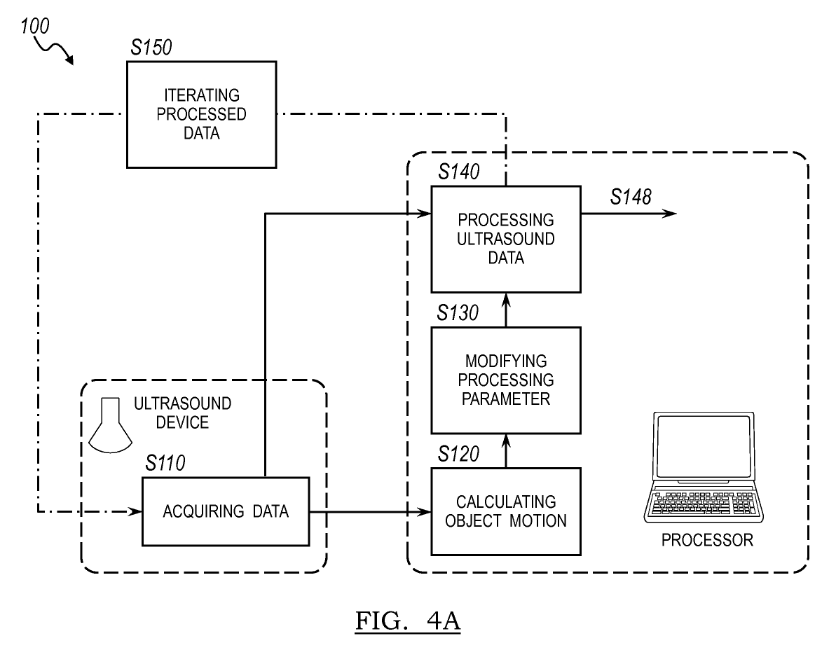

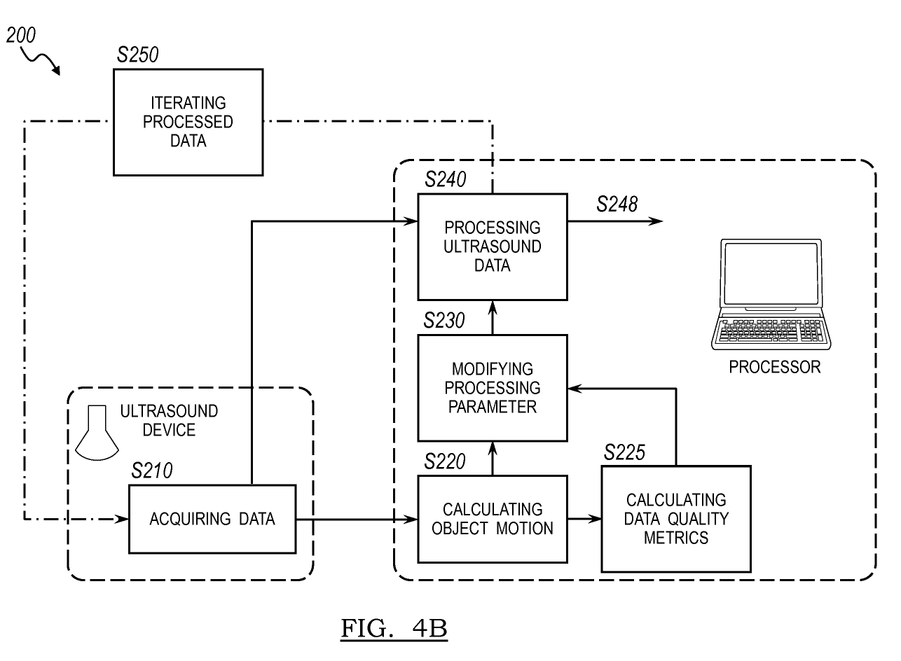

As shown in FIGS. 4A and 4B, the method 100 or 200 may additionally include the step of iterating the processed data S150 or S250. Step S150 is preferably implemented in method 100 in substantially the same way as Step S250 is implemented in method 200. Iterating processed data functions to repeat the processing steps to refine a final data output. Calculating object motion, calculating DQM, modifying processing parameters, processing data, and/or additional or alternative steps are preferably repeated using the output from the data processing as the input data (preferably in place of the acquired data). Alternatively, the input data itself may be modified based on the output from processing the ultrasound data S140. In this method, the acquired data or the processing of the acquired data is preferably modified at least one time, but any number of iterations may alternatively be performed. Iterating the processed data preferably improves the calculation of object motion compared to a previous calculation of object motion. Thus, in method 200 the improved object motion calculation preferably improves the data processing step. DQM information may additionally be used to determine processing operations for particular areas of ultrasound data. The DQM is preferably used to determine areas of greater interest and areas of lesser interest, such as by distinguishing between tissue and blood. This can be used to create an adaptive resolution ultrasound image. Higher resolution processing is preferably performed in areas of greater interest while lower resolution processing is performed in areas that are of lesser interest.

### 4. Dynamic Processing To Control An Outside Device

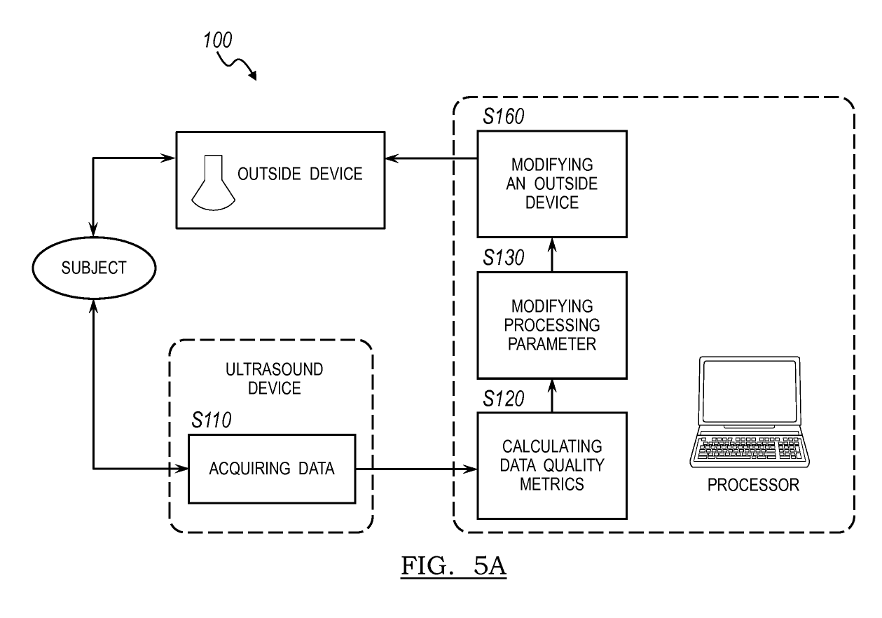

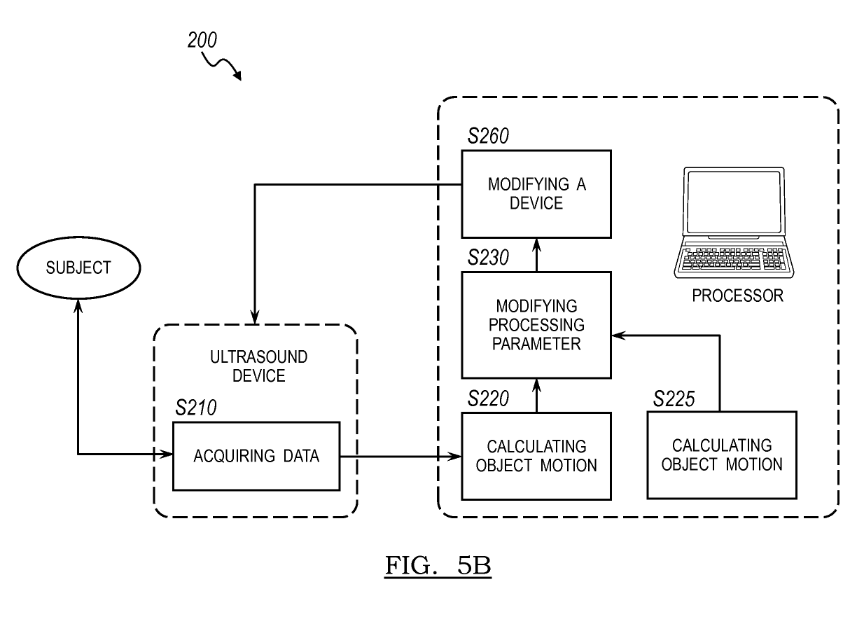

As shown in FIGS. 5A and 5B, the method 100 or 200 of dynamic ultrasound processing may alternatively and/or additionally include modifying an outside device S160 or S260. Step S160 is preferably implemented in method 100 in substantially the same way as Step S260 is implemented in method 200. Step S160 is preferably used in place of Step S140 (e.g., Step S140 is responsible for generating the modification instructions for the outside device), but may alternatively be used in parallel with Step S140, may depend upon results from Step S140, and/or be used with any suitable combination of other suitable steps. Additionally, multiple devices may have parameters modified based on object motion calculations. Step S160 functions to control a device using a parameter controlled by object motion measurements. A parameter of the outside device operation is preferably dependent upon the tissue motion calculation, or alternatively, multiple parameters may be dependent upon the tissue motion calculation. In one variation of method 200, the position or operation of an ultrasound device, or probe, is preferably modified to maximize DQM, which would preferably act as an indicator of the quality of the acquired data. The outside device additionally may interact with a subject such as a patient or more specifically, tissue of a patient. The subject may additionally be the tissue interrogated by the 3D ultrasound device. As an example, Step S160 may be used to gate the data acquisition of a secondary diagnostic device such as a Positron Emission Tomography (PET), Magnetic Resonance Imaging (MRI), or Computed Tomography (CT) based on tissue motion, to reduce motion based data degradation or synchronize acquisition with physiological events (e.g., breathing or heart motion). As another example, Step S160 may be used in guidance of a high intensity focused ultrasound (HIFU) for tissue ablation or heating. Beam shape and energy may be altered based on tissue motion to optimize the ablation therapy. The outside device may alternatively be any suitable medical device.

### 5. Dynamic Processing of Ultrasound Motion Data

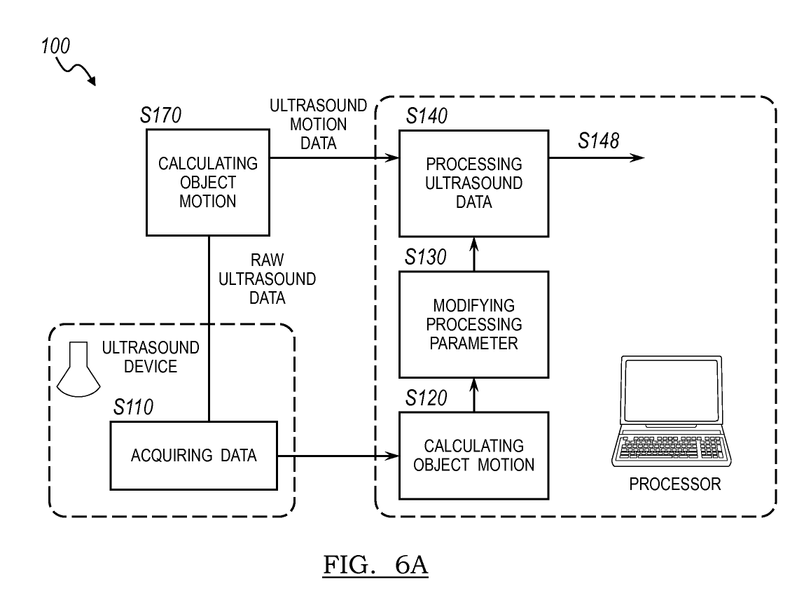

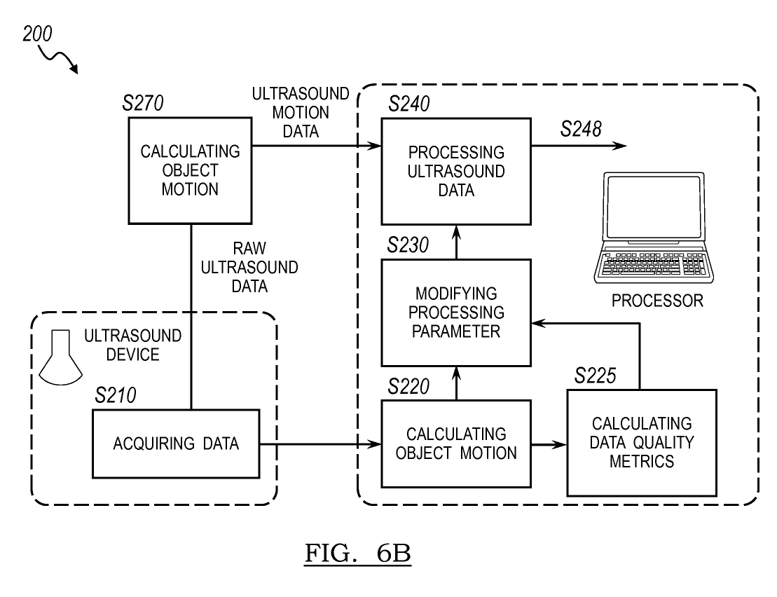

In an additional alternative shown in FIGS. 6A and 6B, the method 100 or 200 may include calculating object motion from raw ultrasound data S170 or S270. Step S170 is preferably implemented in method 100 in substantially the same way as Step S270 is in method 200. Step S170 functions to calculate ultrasound motion data to use as the ultrasound data used in Step S140. The ultrasound motion data is preferably a measurement of tissue velocity, displacement, acceleration, strain, strain rate, or any suitable characteristic of probe, tissue motion, or tissue deformation. The ultrasound motion data may additionally or alternatively be correlation functions, matching functions, or Doppler group (packet) data. In this variation, ultrasound motion data is used as the ultrasound data during Step S140. The object motion calculation is preferably acquired from ultrasound data using speckle tracking, Doppler, block matching, and/or any suitable tracking technique. Step S170 is preferably substantially similar to Step S120. In one variation, Step S120 and S170 are performed in the same step with the results being used to modify a processing parameter and as the ultrasound data to be processed.

### 6. A System For Dynamic Processing

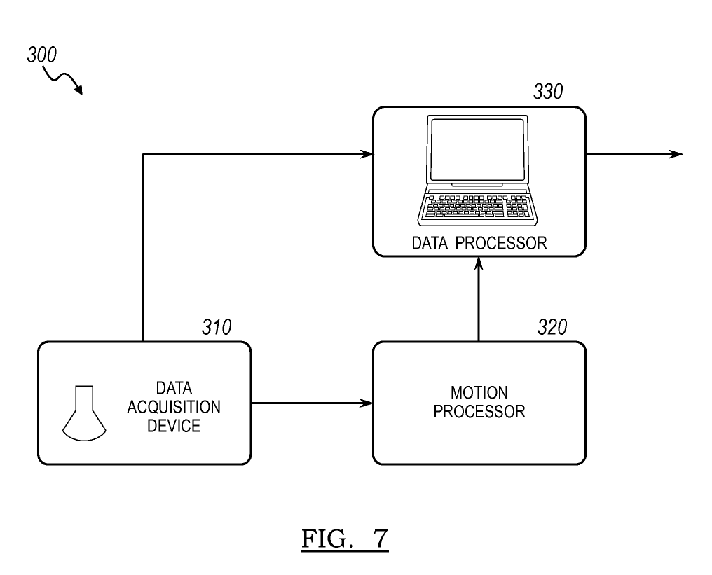

As shown in FIG. 7, the system 300 of the preferred embodiment includes an ultrasound data acquisition device 310, a motion processor 320, and a data processor 330. The system functions to substantially implement the above methods and variations. The ultrasound data acquisition device is preferably a data input, but may alternatively be an ultrasound transducer, an analog to digital converter, a data buffer, data storage device, data processor (to format raw ultrasound data), and/or any suitable device that can function as an ultrasound data source. The motion processor 320 functions to calculate the object motion from the ultrasound data. The motion processor may additionally calculate the DQM but an additional device may alternatively perform the DQM calculation. The data processor functions to convert the ultrasound data into another form of data using the object motion information and/or the DQM as parameter inputs to determine the processing parameters. The system 300 may alternatively be implemented by any suitable device, such as a computer-readable medium that stores computer readable instructions. The instructions are preferably executed by a computer readable components for executing the above method of dynamically processing ultrasound data. The computer-readable medium may be stored on any suitable computer readable media such as RAMs, ROMs, flash memory, EEPROMs, optical devices (e.g., CD or DVD), hard drives, floppy drives, or any suitable device. The computer-executable component is preferably a processor but the instructions may alternatively or additionally be executed by any suitable dedicated hardware device.

As a person skilled in the art will recognize from the previous detailed description and from the figures and claims, modifications and changes can be made to the preferred embodiments of the invention without departing from the scope of this invention defined in the following claims.

We claim:

1. A method for transforming ultrasound data comprising:
acquiring ultrasound data;
calculating object motion from the collected ultrasound data;
modifying a processing parameter using parameter inputs related to the calculated object motion;
processing ultrasound data related to the acquired ultrasound data according to the processing parameter; and
outputting the processed ultrasound data.

2. The method of claim 1, wherein the step of processing includes forming an ultrasound image from the acquired ultrasound data, resampling an ultrasound image, and performing temporal processing.

3. The method of claim 2, wherein the temporal processing includes the process of temporal integration.

4. The method of claim 2, further comprising calculating a data quality metric (DQM) from the calculated object motion, wherein the parameter inputs include the DQM.

5. The method of claim 4, wherein the parameter inputs include the calculated object motion.

6. The method of claim 4, wherein the step of calculating object motion includes performing speckle tracking.

7. The method of claim 1, further comprising calculating a data quality metric (DQM) from the calculated object motion, wherein the parameter inputs include the DQM.

8. The method of claim 7, wherein the parameter inputs include the calculated object motion.

9. The method of claim 7, wherein the step of calculating object motion includes performing speckle tracking.

10. The method of claim 7, wherein the parameter inputs additionally includes the calculated object motion, and wherein the step of modifying a processing parameter includes modifying a first processing parameter using the calculated object motion and modifying a second processing parameter using the DQM.

11. The method of claim 10, wherein the first parameter affects the resampling coefficients used to resample an ultrasound image during the processing of the ultrasound data and the second parameter affects the image processing process during the processing of the ultrasound data.

12. The method of claim 1, wherein the step of calculating object motion includes performing speckle tracking.

13. The method of claim 12, comprising calculating a data quality metric (DQM) from a cross correction during speckle tracking, wherein the DQM is a data quality index (DQI).

14. The method of claim 13, further comprising sorting data according to the DQI.

15. The method of claim 14, wherein the step of sorting data according to the DQI includes differentiating between pixels of different DQI values and determining the processing of the pixels according to the differentiation.

16. The method of claim 1, wherein the step of processing includes processing the acquired ultrasound data.

17. The method of claim 1, wherein the step of processing includes processing the calculated object motion data.

18. The method of claim 1, further comprising modifying an outside device according to the outputted processed ultrasound data, wherein the processing of ultrasound data includes calculating the modifications of the outside device.

19. The method of claim 1, further comprising repeating the steps of calculating object motion, modifying a processing parameter, and processing the ultrasound data, before outputting the ultrasound data.

20. A system for handling ultrasound data comprising:
an ultrasound acquisition device for collecting ultrasound data;
a motion processor that calculates object motion from the ultrasound data; and
a data processor that determines processing parameters from calculations from the motion processor and processes the ultrasound data supplied by the ultrasound acquisition device.

21. The system of claim 20, further comprising an output device for outputting the processed ultrasound data.

22. The system of claim 20, wherein the motion processor additionally produces a data quality metric (DQM) and the data processor uses the DQM to determine the processing parameters.
